(19) United States
(12) Patent Application Publication
(10) Pub. No.: US 2003/0113281 A1
(43) Pub. Date: Jun. 19, 2003

(54) Assay for identification of compounds that promote melanin production and retinoid-like compounds identified by said assay

(75) Inventor: Deepa Rungta, San Diego, CA (US)

Correspondence Address: BURNS, DOANE, SWECKER & MATHIS, L.L.P., P.O. Box 1404, Alexandria, VA, 22313-1404, US

(73) Assignee: Galderma Research & Development, S.N.C., Valbonne (FR)

(21) Appl. No.: 10/346,054

(22) Filed: Jan. 17, 2003

Related U.S. Application Data
(63) Continuation of application No. 09/673,974, filed on Oct. 25, 2000, now abandoned.
Application No. 09/673,974 is a 371 of international application No. PCT/US99/09845, filed on May 6, 1999.
(60) Provisional application No. 60/084,478, filed on May 6, 1998.

(51) Int. Cl. A61K 7/42; A61K 7/06; A61K 7/13

(52) U.S. Cl. 424/59; 8/405

(57) ABSTRACT

An in vitro assay for selecting compounds that alter pigmentation of skin is provided. Also, a novel class of pro-pigmentatory compounds is provided which comprise substituted aromatic or heterocyclic carboxylic acids, or derivatives thereof, or pharmaceutically acceptable salts, which do not contain a pheno, naphthol, thiophenol, or a thionaphthol function in free or protected form. In a preferred embodiment, thess compounds will display activity for RXRs. These compounds may be used for altering pigmentation of human skin and/or hair in cosmetic or dermatological compositions, and for the treatment of disorders and disease conditions that affect skin or hair pigmentation.

### BACKGROUND OF THE INVENTION

Normal skin color is the result of the combined expression of an admixture of different pigments of which melanin, a brown pigment, is the major component. Melanin is synthesized by specialized cells, melanocytes, which are found within the epidermis. Melanocytes synthesize melanin within organelles, which are called melanosomes. As the melanosomes become saturated with melanin, they are transported to the dendritic arms of the melanosomes and are transferred to the surrounding keratinocytes. The keratinocytes then migrate to the surface of the skin causing the skin to exhibit a brown pigmentation. The amount of melanin in keratinocytes determines the extent of pigmentation in the skin and hair.

Because of melanin's effect on skin and hair pigmentation, research has been conducted which is targeted toward identifying compounds that affect melanin production and/or transport of melanin to the surrounding keratinocytes. Such compounds have potential application for altering hair and skin pigmentation. For example, it is known that melanocyte stimulating hormone (MSH), and compounds such as theophylline, induce pigmentation. Further, analogs of MSH are being tested for clinical efficacy for promoting skin pigmentation. Also, it has been reported that alpha-lipoproteins can stimulate melanin production in the skin. (Jpn. Kokai Tokyo, Koho, JP 82-153348, GB 2124900, by Empresa Cubana Importadora y Exportadora de Productos Medicos, Cuba.) Conversely, the use of some compounds to inhibit pigmentation, e.g., for treating melasma is also known, e.g., hydroquinone (HQ), and monobenezene ether (Pathak et al, J. American Academy Dermotol. 15:894-899 (1986); Smith et al, Pigment Cell Research, 1:386-389 (1988)), and arbutin, a gluco conjugate of HQ (Proceeding Japan Soc. Invest. Dermotol. 12:138-139 (1988)).

Moreover, it has been reported that a kojic acid and derivatives thereof and/or valeric acids inhibit melanin formation (Japan Kokai Tokyo Koho, JP 03011010; Oyama, Yasuaki, Sansei Pharmaceutical Co., Ltd., Japan.)

Further, it has been reported that propionic, butyric, and valeric acids and their salts, in combination with or without unsaturated fatty acids, suppress the formation of tyrosinase, which catalyzes production of melanin (Mishima et al, EPA 345081, Hayashibara Biochemical Laboratories, Inc., Japan.)

EP 0 509 241 and WO 93/19729 describe that the hyperpigmentation, or tanning the skin, can be carried out by topically applying thereto an efficient amount of trans-retinoic acid for a sufficient length of time. Nevertheless, the compounds used in those methods induce irritation.

An in vitro method purportedly suitable for identifying compounds that inhibit de novo melanin synthesis and potential anti-melanosoma, cytotoxic compounds was reported by Dooley et al, Pharmcol., 7:188-200 (1994). This method utilizes two immortalized murine cell line of melanocyte origin, Mel-Ab (Dooley et al, Oncogene, 3:531-535 (1988)), and the other of fibroblast origin. Mel-Ab cells are derived from a spontaneously immortalized murine C57BL\6 melanocyte line that grows rapidly in culture and produces copious amounts of melanin. This assay was reportedly used to screen compounds having similar structure to intermediates in the melanin biosynthetic pathway which are unique to melanocytes. In particular, para-substituted phenols related to tyrosine and 1,2-dihydroxybenzene related to DOPA were screened, as well as 1,3-dihydroxybenzenes, 1,4-dihydroxybenzenes, 1,4-dimethoxybenzenes, and 1,4-benzoquinones. Several compounds that purportedly alter pigmentation were identified using this screen. However, unlike the present invention, this in vitro screen instead utilizes immortalized murine cell lines and not normal human melanocytes.

However, it would be beneficial if other compounds that effect pigmentation and melanin production and/or transport could be identified. It would also be beneficial if compounds could be identified which are well tolerated and non-irritating to the skin. Moreover, it would be useful if an improved in vitro screening assay were developed which provided for the identification of such compounds.

### BRIEF DESCRIPTION AND OBJECTS OF THE INVENTION

Toward that end, it is an object of the invention to provide a novel assay for the identification of compounds that can alter (enhance or inhibit) the expression of melanin by melanocytes, preferably human melanocytes.

It is another object of the invention to provide dermatological/cosmetic/pharmaceutical compositions comprising a melanin-promoting or inhibiting amount of at least one compound identified by such assay.

It is a more specific object of the invention to provide topically applicable dermatological/cosmetic/pharmaceutical compositions comprising a melanin-enhancing or inhibiting amount of at least one compound that alters (enhances or inhibits) melanin production by human melanocytes.

It is an even more specific object of the invention to provide dermatological/cosmetic/pharmaceutical compositions comprising a melanin-enhancing or inhibiting amount of at least one substituted aromatic or heterocyclic carboxylic acid or a derivative thereof with the proviso that said carboxylic acid does not comprise a free phenol, naphthol, thiophenol, or thionaphthol functional group, or one which is protected by a protective group.

It is another specific object of the invention to provide a method of altering the pigmentation of the skin and/or hair in a subject in need of such treatment comprising topically applying an effective amount of a composition comprising at least one melanin-affecting (increasing or inhibitory) retinoid compound for a sufficient length of time to induce skin and/or hair pigmentation.

It is a more specific object of the invention to provide a method of altering, either increasing or decreasing, pigmentation of the skin and/or hair, in a subject in need of such treatment, comprising topically applying an effective amount of a composition comprising at least one substituted aromatic or heterocyclic carboxylic acid or a derivative thereof for a sufficient length of time to affect skin and/or hair pigmentation, with the proviso that said carboxylic acid does not comprise a free or protected phenol, naphthol, thiophenol, or thionaphthol group.

### BRIEF DESCRIPTION OF THE FIGURES

FIG. 1 contains the structures for compounds identified according to the invention which affect melanin production; and

FIGS. 2 through 4 list pigmentary diseases that may be treated using the compounds of the invention.

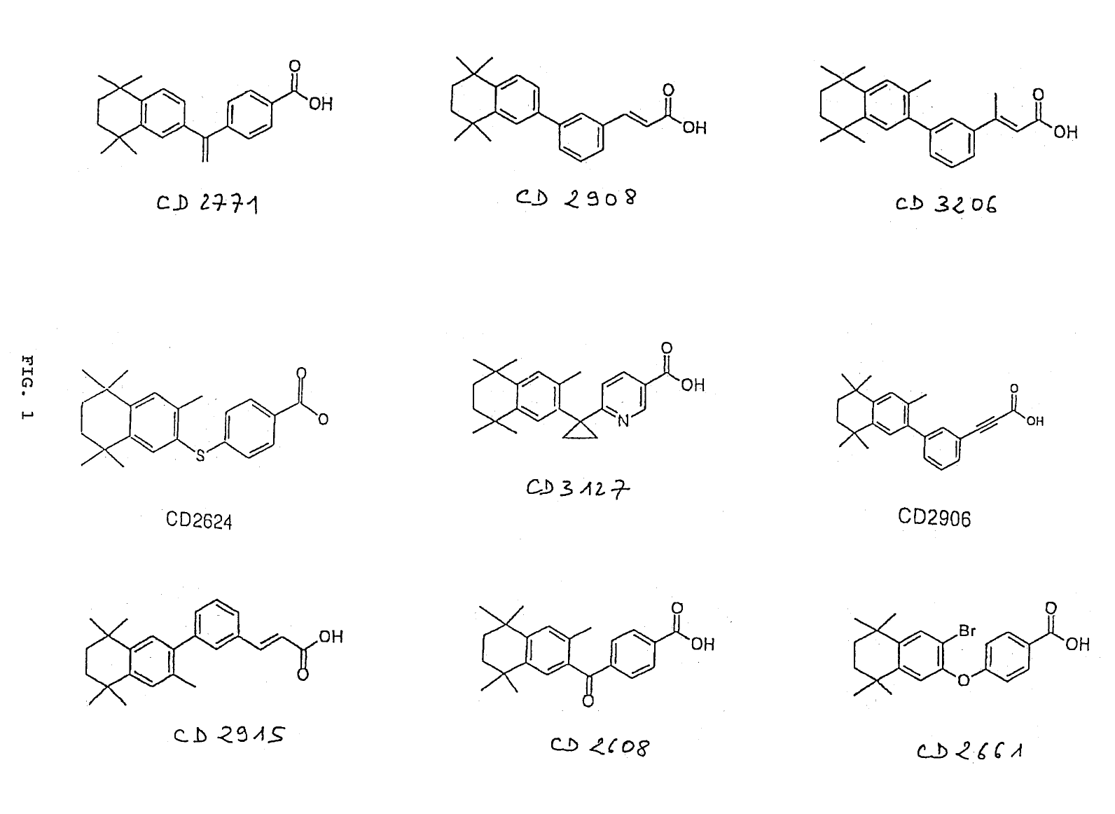

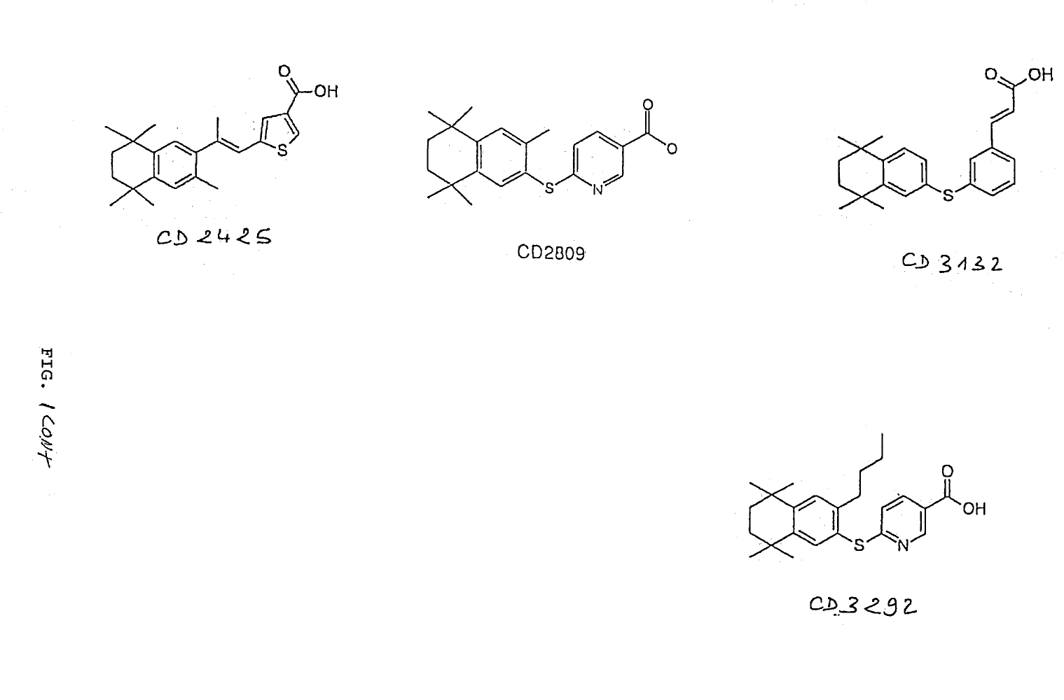

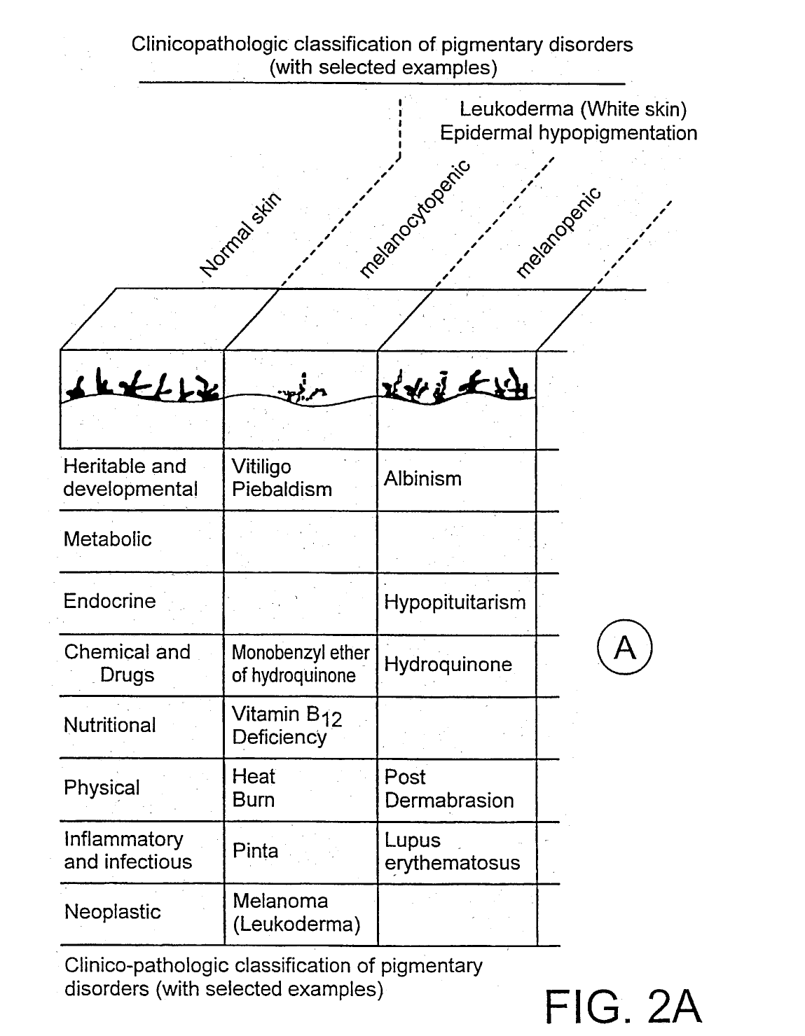

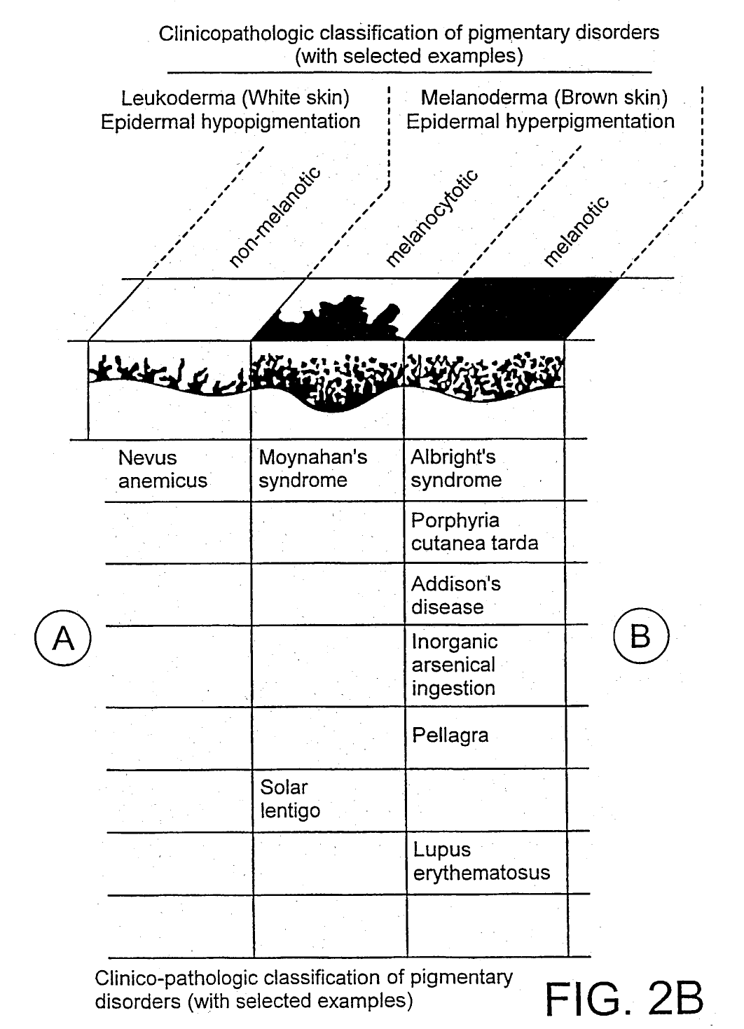

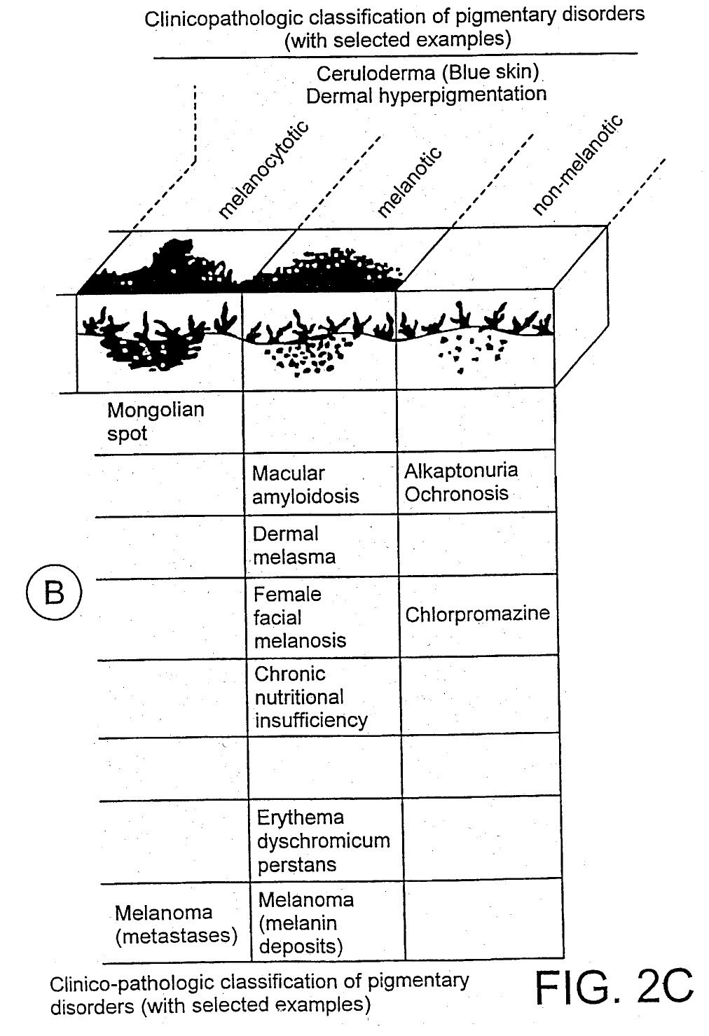

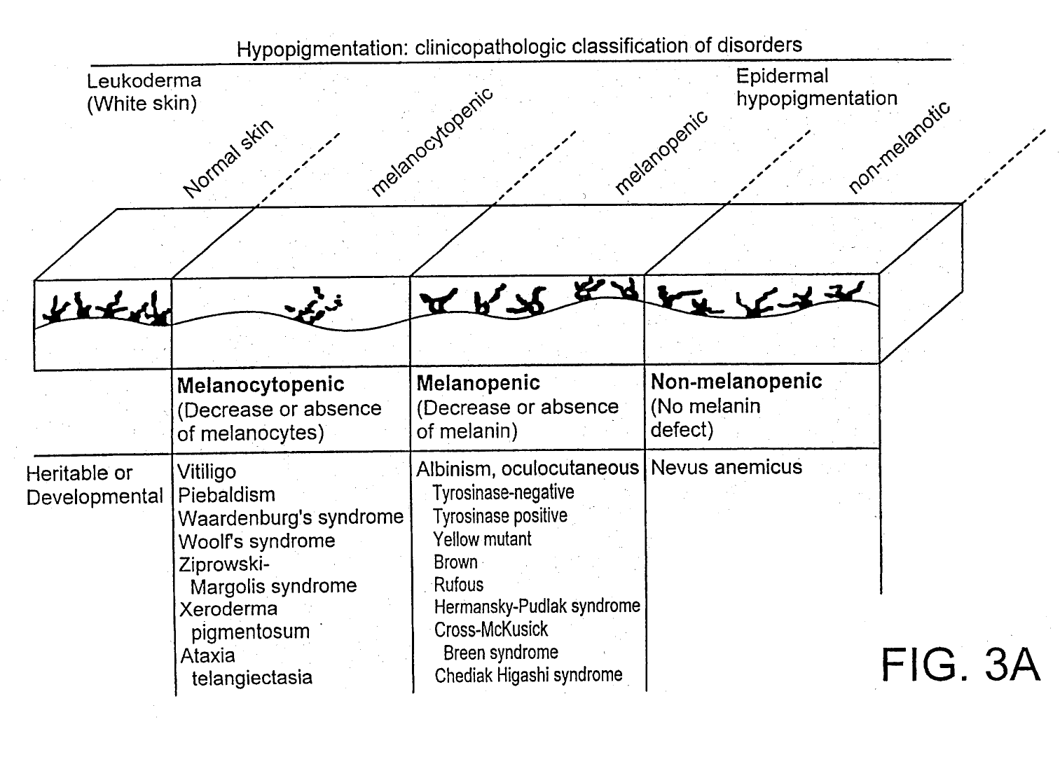

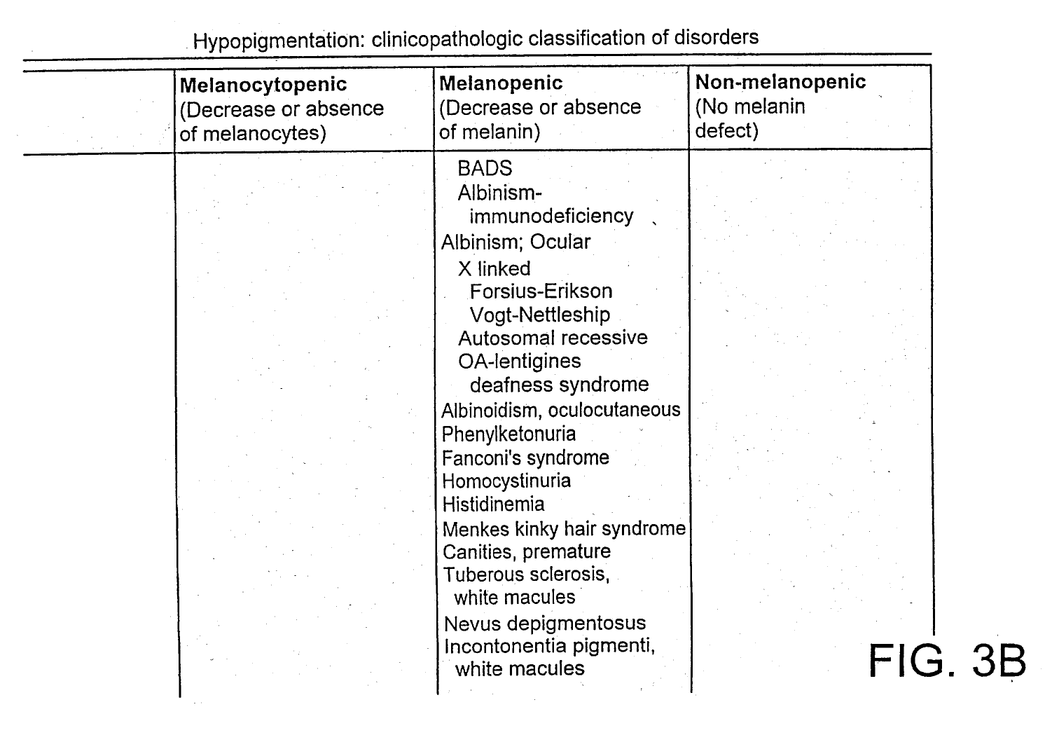

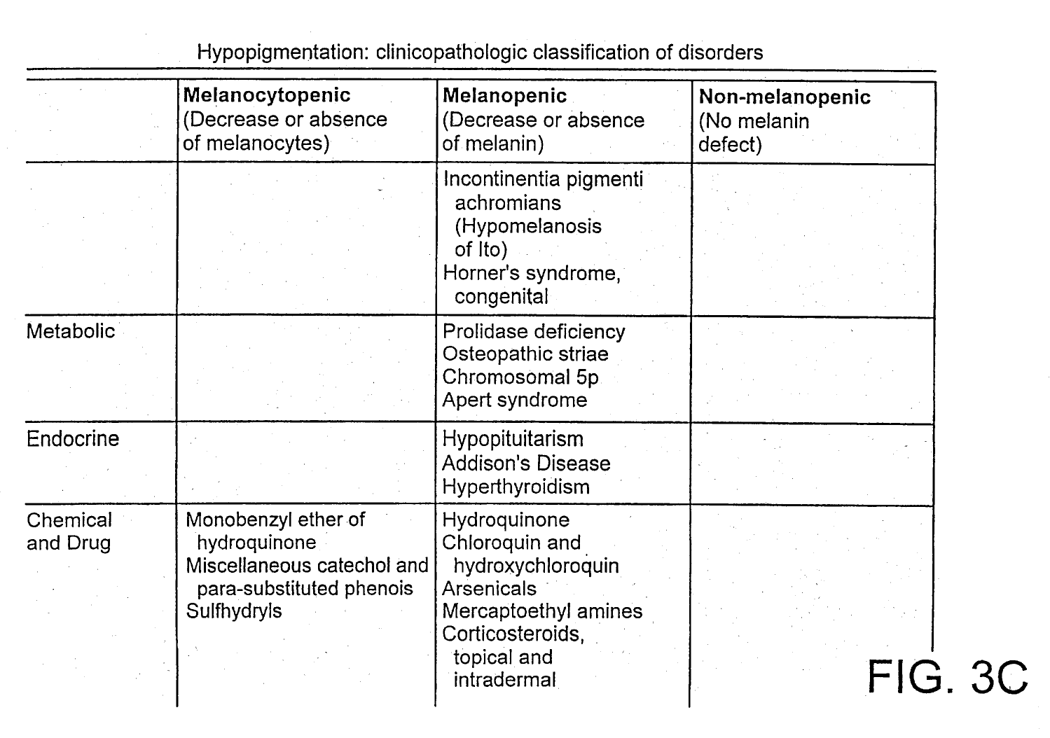

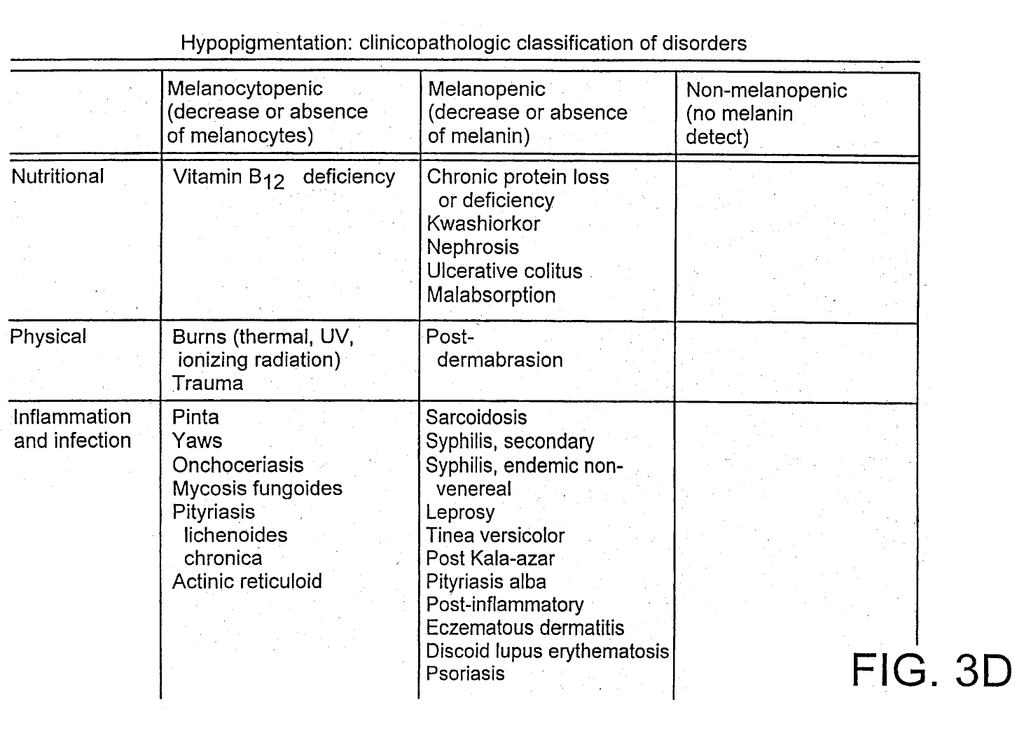

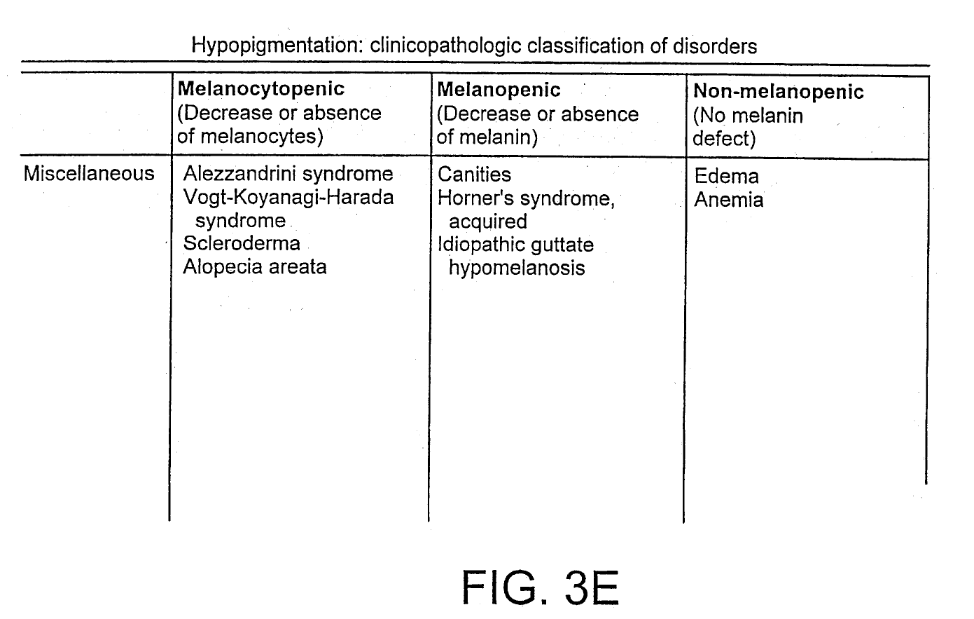

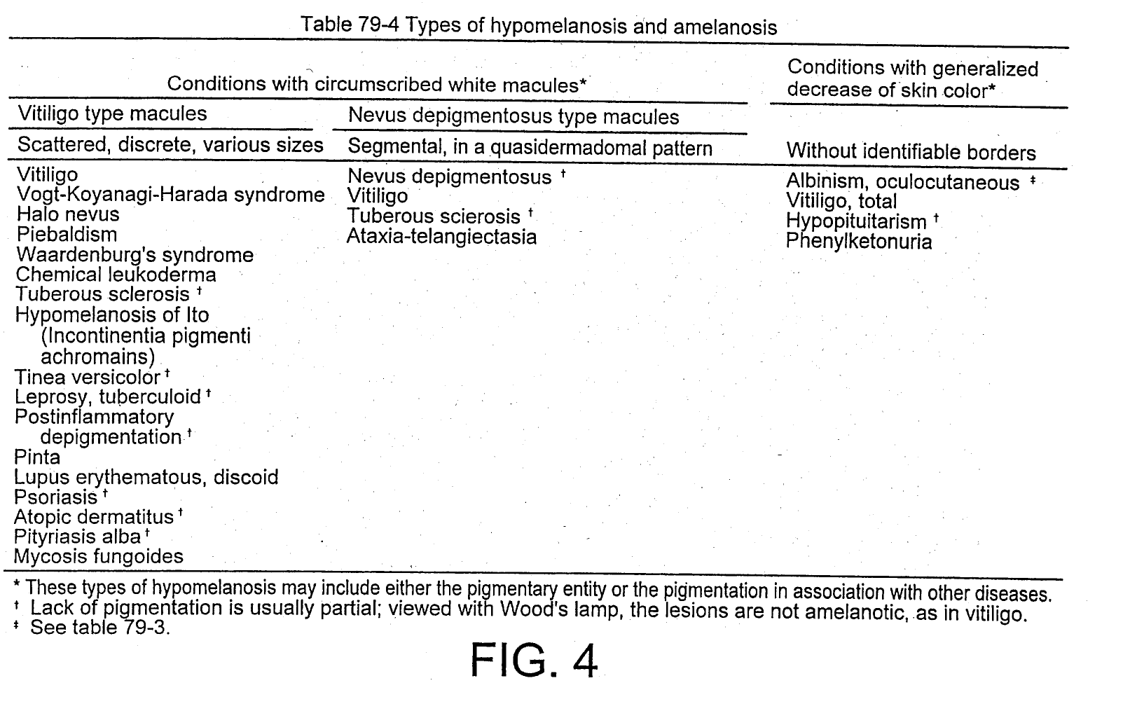

### DETAILED DESCRIPTION OF THE INVENTION

In a first embodiment, the present invention provides a novel assay for identifying compounds that alter (increase or inhibit) the expression of melanin by human melanocytes, preferably compounds that increase melanin production by human melanocytes.

In general, the subject assay will test the effect of a particular compound or combination of compounds on the growth or proliferation of human melanocytes in culture, and also the effect of said same compound on melanin production by said cultured human melanocytes, normalizing melanin content based on the number (proliferation) of said cultured human melanocytes, and identifying a compound or compounds which affects melanin production based on an increase or decrease in melanin production per cell.

More specifically, the subject assay will comprise:

(i) contacting at least two human melanocyte cultures with at least one compound to be screened for its effect on melanin production;

(ii) measuring the proliferation of melanocytes contained in one of said human melanocyte cultures;

(iii) measuring the amount of melanin contained in a second human melanocyte culture;

(iv) comparing the data obtained in steps (ii) and (iii) in order to determine the effect of such compound or compounds on melanin production (increase or decrease), and normalizing the data based on the number of melanocytes contained in said cultures;

(v) further comparing the data obtained in steps (ii) and (iii) to the proliferation and melanin production in control human melanocyte cultures which are cultured under identical conditions as the previous cultures except in the absence of said compound; and

(vi) identifying a screened compound as one that alters (increases or decreases) melanin expression by human melanocytes based on an increase or decrease in melanin production by melanocytes cultured in the presence of said compound relative to control cultures not contacted with said compound.

This method will preferably be effected by obtaining human melanocytes, e.g., neonatal skin melanocytes, from a suitable source. These melanocytes are then seeded simultaneously into two cell culture chambers, e.g., a 96-well plate and a 24-well plate. After such cells have been permitted to adhere to the surface of the wells, the respective wells are then incubated with a fixed amount of a particular compound or compounds, the effect of which on melanin expression is to be determined. Such incubation will be effected for a time period sufficient to evaluate the effect, if any, of such compound or compounds on melanin production by such cultured melanocytes.

The incubation then will vary from about one minute to about one month, more preferably from about one hour to about two weeks, and most preferably from about 12 hours to one week. After incubation, the proliferation of cells in one of said cultures is then evaluated. This can be effected by suitable methods, e.g., by absorbence.

In a preferred embodiment, the cells in the 96-well plate are treated with 3-(4,5-dimethylthiazol-2-yl)-5-(3-carboxymethoxyphenyl)-2-(4-sulfophenyl)-2H-tetrazolium (a tetrazolium dye conventionally used to colormetrically determine proliferation of cells) (MTS) and the absorbence of the plate read at 490 nm to determine the proliferation of cells in said 96-well plate. These results are then compared to a control melanocyte culture which is cultured under the same conditions, and subsequently treated with MTS solution, but which is not treated with the test compound.

The second culture, e.g., a 24-well plate, containing a human melanocyte culture which has been incubated with the test compound under the same conditions, is then tested to determine the amount of melanin expressed by cells contained therein. This can be effected by known methods. In the present invention, this will preferably be effected by washing such cells, e.g., with phosphate buffered saline (PBS), lysing said washed cells with an alkaline solution, e.g., with 1N NaOH, and measuring the amount of alkali soluble melanin contained therein, e.g., by measuring absorbence of alkali soluble melanin at 405 nm. This data is then preferably compared to a control melanocyte culture, grown under identical conditions but in the absence of said tested compound.

The data obtained from both melanocyte cell cultures is then normalized based on melanocyte proliferation (cell number) in order to determine the effect, if any, of the particular compound or compounds on melanin production and accumulation of melanin by melanocytes.

Compounds which alter melanin expression according to the invention are those compounds which affect melanin production and accumulation in cultured human melanocytes. Preferably, such compound will alter melanin expression by at least 10% relative to control melanocyte cultures. More preferably, such compound will affect melanin production by at least 20%. Most preferably, the compound will affect melanin production and accumulation on the order of 150 to 250%, or greater.

Therefore, the subject invention is directed to the identification of compounds or combinations thereof that increase or decrease melanin production and accumulation by human melanocytes. Compounds which increase melanin production and accumulation can be used, e.g., to promote tanning or browning of skin, and to treat or prevent graying of hair. Also, such compounds can be used to treat diseases or conditions associated with hypopigmentation, e.g., vitiligo.

Compounds which decrease melanin production can be utilized for treatment of subjects having diseases or conditions associated with hyperpigmentation. Such diseases and conditions include melasma or age-related hyperpigmentation, and chloasma.

Also, compounds which inhibit melanin production and/or accumulation may be used to treat and/or prevent hyperpigmentation associated with aging, e.g., “liver spots” often observed on the hands and face of older persons. A list of disease and conditions associated with pigmentation disorders may be found in Dermatology in General Medicine (Fitzpatric, T. B., et al) which are incorporated by reference in their entirety herein.

The compounds of the present invention can be useful in the treatment of hypopigmentation afflications such as vitiligo, post-inflammatory hypopigmentations or depigmentations, for the treatment of hypopigmentations or depigmentations after skin grafts, for the treatment of hypopigmentations or depigmentations due to overexposure to ultraviolet rays, for treating post-cicatrisation hypopigmentations or depigmentations, or for treating hypopigmentations or depigmentations due to aging or lentigo.

Compounds which decrease melanin production can be useful in the treatment of melasma or age associated hyperpigmentation.

Further, the subject assay can be used to identify combinations of compounds that affect (increase or decrease) melanin production or accumulation by human melanocytes, in particular combinations having synergistic effects on melanin production or accumulation.

As discussed above, in a second embodiment, the present invention is directed to pharmaceutical/dermatological/cosmetic compounds that contain at least one melanin-affecting compound according to the invention. In this regard, a novel class of compounds has been discovered using the subject assay methods which promote melanin production and/or accumulation by normal human melanocytes. These compounds are advantageous also in that they are well tolerated and non-irritating to the skin. This is surprising because many other retinoid compounds screened using the subject assay did not affect pigmentation under similar conditions. These compounds are hypothesized to affect melanin production and/or accumulation via the retinoid signaling pathway.

In a preferred embodiment, the present invention provides pharmaceutical/cosmetic/dermatological compounds that comprise an amount of at least one substituted aromatic or heterocyclic carboxylic acid, or a derivative thereof, with the proviso that such carboxylic acid does not contain a phenol, naphthol, thiophenol, or thionaphthol function, in free or protected form. Such carboxylic acids include, by way of example, benzoic acid, propiolic acid, nicotinic acid, acrylic acid, butenoic acid and naphtoic acid.

In a preferred embodiment, the substituted aromatic or heterocyclic carboxylic acids of the present invention will display activity for RXRs. According to the present invention a compound displaying RXR activity is a compound which exhibits a binding for RXR less than 5000 nM, and preferably less than 1000 nM. The determination of the binding for RXRs is well known by those skilled in the art and is reported in, for example: (1) “Selective Synthetic Ligands for Nuclear Retinoic Acid Receptor Subtypes” in RETINOIDS, Progress in Research and Clinical Applications, Chapitre 10 (pp 261-267), Marcel Dekker Inc, published by Maria A. Livrea et Lester Packer; (2) Synthetic Retinoids: Receptor Selectivity and Biological Activity” in Pharmacol Skin, Basel, Karger, 1993, Vol. 5, pp 117-127; (3) “Selective Synthetic Ligands for Human Nuclear Retinoic Acid Receptors” in Skin Pharmacology, 1992, Vol. 5, pp 57-65; (4) “Identification of Synthetic Retinoids with Selectivity for Human Nuclear Retinoic Acid Receptor-γ” in Biochemical and Biophysical Research Communications, Vol. 186, No. 2, July 1992, pp 977-983; and (5) “Selective High Affinity RAR-α or RAR-β Retinoic Acid Receptor Ligands” in Mol. Pharmacol., Vol. 40, pp 556-562.

Also, in a more preferred embodiment, the compound displaying activity for RXRs exhibits an agonsit activity for RXRs. This agonist activity for RXRs may be determined for instance by the method reported in U.S. Pat. No. 5,696,104, the entire contents of which are hereby incorporated by reference.

More specifically, compounds which have been surprisingly discovered to promote melanin production and/or accumulation by human melanocytes according to the invention, include:

4-[1-(5,6.7,8-tetrahydro-5,5,8,8-tetramethyl-2-naphthyl)ethenyl]benzoic acid (CD No. 2771);

3-[3-(5,5,8,8-tetramethyl-5,6,7,8-tetrahydro-naphthalen-2-yl)-phenyl]-acrylic acid (CD No. 2908);

3-[3-(3,5.5,8,8-pentamethyl-5,6,7,8-tetrahydro-naphthalen-2-yl)-phenyl]-but-2-enoic acid (CD No.3206);

6-[1-(3,5,5,8,8-pentamethyl-5,6,7,8-tetrahydro-naphthalen-2-yl)-cyclopropyl]-nicotinic acid (CD No. 3127);

3-[3-(3,5,5,8,8-pentamethyl-5.6,7.8-tetrahydro-naphthalen-2-yl)-phenyl]-acrylic acid (CD No. 2915);

4-(3,5,5,8,8-pentamethyl-5,6,7,8-tetrahydro-naphthalene-2-carbonyl)-benzoicacid (CD No. 2608);

4-(3-bromo-5,5,8,8-tetramethyl-5,6,7,8-tetrahydro-naphthalen-2-yloxy)-benzoic acid (CD No. 2661);

5-[(E)-2-(3,5,5,8,8-pentamethyl-5,6,7,8-tetrahydro-naphthalen-2-yl)-propenyl]-thiophene-3-carboxylic acid (CD No. 2425);

3-[3-(5,5,8,8-tetramethyl-5,6,7,8-tetrahydro-naphthalen-2-ylsulfanyl)-phenyl]-acrylic acid (CD No.3132);

6-(3-butyl-5,5,8,8-tetramethyl-5,6,7,8-tetrahydro-naphthalen-2-ylsulfanyl)-nicotinic acid (CD No.3292);

4-(3,5,5,8,8-pentamethyl-5,6,7,8-tetrahydro-2-naphthylthio) benzoic acid (CD No. 2624);

3-(3,5,5,8,8-pentamethyl-5,6,7,8-tetrahydro-2 naphthyl)phenyl propiolic acid (CD No. 2906); and

6-(3,5,5,8,8-pentamethyl-5,6,7,8-tetrahydro-2 naphthylthio) nicotinic acid (CD No. 2809).

As noted above, the pharmaceutical/dermatological/cosmetic compositions will be in a form suitable for topical application to the skin and/or hair. In a preferred embodiment, these compositions will be in a form suitable for artificial tanning and/or browning of the skin.

These compositions may comprise other pharmaceutically, cosmetically, or dermatologically acceptable constituents typically formulated into these types of compositions, such as ionic or non-ionic thickeners, softeners, antioxidants, opacifiers, stabilizers, emollients, organic sunscreens which are active in the UV-A and/or UV-B region, photoprotective inorganic pigments, and non-pigments, moisturizers, vitamins, fragrances, preservatives, fillers, sequestering agents, dyestuffs, and colorants.

Naturally, one skilled in the art should take care to select this or these optimal complementary compounds and/or the amounts thereof such that the advantageous properties intrinsically associated with the compositions of the invention are not adversely affected by the addition or additions envisioned.

The inventive compositions may comprise one or more compounds that affect melanin production and/or accumulation by melanocytes, e.g., synergistic combinations. Also, such compositions may comprise other compounds which promote or inhibit pigmentation, e.g., dihydroxyacetone (DHA), or derivatives thereof. Suitable compounds are disclosed in U.S. Ser. No. 08/819,084, filed Mar. 18, 1997 by Tuloup et al, and assigned to L'ORÉAL. Also, MSH and analogs thereof which promote pigmentation may be included. Conversely, compounds which inhibit pigmentation such as HQ, monobenzene ether and arbutin may be included with compounds identified using the subject assay that inhibit pigmentation.

The subject compositions will be in a form suitable for topical application to human skin and/or hair. Suitable forms include a cream, a milk, a cream-gel, a fluid lotion, an oil-in-water emulsion or water-in-oil emulsion, a vesicle dispersion, or any other form typically employed in the art.

The amount of the at least one melanin-affecting compound contained in the subject compositions will typically range from about 0.0001% to about 30% by weight relative to the total weight of the composition, and preferably will range in concentration from about 0.5% to about 15% by weight relative to the total weight of the composition, and more preferably will range in concentration from about 0.001% to 5% by weight relative to the total weight of the composition.

The present invention also encompasses a regimen of treatment, cosmetic, dermatological, or therapeutic, comprising topical application of an amount of a melanin-affecting compound according to the invention. An effective amount is an amount sufficient to affect (increase or decrease) pigmentation of the skin and/or hair. Such regimen may be effected in conjunction with other compounds that affect pigmentation of the skin and/or hair. Such regimen can be effected repeatedly until the desired effect on pigmentation is achieved. Chronic administration should be suitable as the subject compounds are not irritating to the skin.

As discussed, the present invention encompasses, in particular, compositions which contain at least one compound according to the invention that are suitable for promoting coloration of the hair. Such compositions may include other constituents typically included in hair formulations, e.g., conditioning agents, pigments and color ingredients, straighteners, permanents, surfactants, perfumes, alcohols, et seq.

In order to better facilitate an understanding of the invention, the following Examples are provided.

### EXAMPLE 1

Normal human melanocytes from neonatal skin were used for the assay. For the primary screening, the cells were seeded simultaneously into 96-well plates and 24-well plates appropriately according to the surface area of the wells, and treated with the test compound for 5 days. The addition of the compound to the plates was done using a Robot to ensure accuracy (same amount in each well). At the end of 5 day treatment, the cells in the 96-well plate were treated with MTS solution and the plate was read at 490 nm to determine the effect of the compounds on the growth of the cells. The cells in the 24-well plates were rinsed with PBS, and then lysed with 1N NaOH, and the absorbence of alkali soluble melanin was measured at 405 nm.

The data from the 96-well plate was analyzed to determine the effect of each compound on the proliferation of the cells, and the data from the 24-well plate was used to determine the effect of the compound on the melanin content of the cells. The melanin content was then normalized to the proliferation of the cells.

Using this method, the inventors have identified the following compounds that increase the melanin content of normal human melanocytes. It is anticipated, based on these results, that other compounds, e.g., retinoids, can be identified which alter (increase or decrease) melanin production by human melanocytes.

| | Maximum Increase in Melanin | |
|---|---|---|
| CD No. | Content (Normalized) | Comment |
| 2771 | 250% at 2 μM | |
| 2908 | 200% at 2 μM | |
| 3206 | 198% at 0.5 μM | |
| 2624 | 181% at 2 μM | |
| 3127 | 179% at 0.1 μM | |
| 2906 | 171% at 0.1 μM | |
| 2915 | 165% at 0.1 μM | |
| 2608 | 157% at 2 μM | |
| 2661 | 155% at 1 μM | |
| 2425 | 149% at 1 μM | |
| 2809 | 145% at 0.5 μM | |
| 3132 | 142% at 2 μM | |
| 3292 | 121% of 1 μM | |

Thus, using the assay of the present invention, the inventors have identified a novel class of pro-pigmentory compounds for normal human melanocytes (increase melanin production thereby). It is hypothesized that these molecules may work through the retinoid signaling pathway. The discovery of a class of retinoid-like molecules having pro-pigmentory activity is surprising especially because other retinoid signaling molecules do not induce pigmentation under similar conditions. This discovery is further advantageous because these compounds are very well tolerated, and are not irritating to the skin. Accordingly, they have potential application in altering pigmentation in skin and/or hair of human subjects.

### EXAMPLE 2

### FORMULATION EXAMPLES

1. Oral Route

(a) The following composition is prepared in the form of a 0.8 g tablet:

| | |
|---|---|
| Compound of formula (2) | 0.005 g |
| Pregelatinized starch | 0.265 g |
| Microcrystalline cellulose | 0.300 g |
| Lactose | 0.200 g |
| Magnesium stearate | 0.030 g |

For the treatment of a post-inflammatory hypopigmentation, 1 to 3 tablets are given to an adult individual per day for three to six months depending on the severity of the case treated.

(b) A drinkable suspension intended for packaging in 5 ml vials is prepared.

| | | |
|---|---|---|
| Compound of formula (4) | 0.050 | g |
| Glycerol | 0.500 | g |
| 70% Sorbitol | 0.500 | g |
| Sodium saccharinate | 0.010 | g |
| Methyl para-hydroxybenzoate | 0.040 | g |
| Flavoring qa | | |
| Purified water qs | 5 | ml |

For the treatment of a hypopigmentation after a skin graft, one vial is given to an adult individual per day for three months depending on the severity of the case treated.

(c) The following formulation intended for packaging in gelatin capsules is prepared:

| | |
|---|---|
| Compound of formula (5) | 0.025 g |
| Corn Starch | 0.060 g |
| Lactose qs | 0.300 g |

The gelatin capsules used consist of gelatin, titanium oxide, and a preserving agent.

In the treatment of vitiligo, one gelatin capsule is given to an adult individual per day for 30 days.

2. Topical Route

(a) The following non-ionic water-in-oil cream is prepared:

| | |
|---|---|
| Compound of formula (8) | 0.100 g |
| Mixture of emulsifying lanolin alcohols, | 39.900 g |
| waxes and refined oils, sold by the | |
| company BDF under the name | |
| “anhydrous Eucerin” | |
| Methyl para-hydroxybenzoate | 0.075 g |
| Propyl para-hydroxybenzoate | 0.075 g |
| Sterile demineralized water qs | 100.000 g |

This cream is applied to a hypopigmented grafted skin once or twice a day for thirty days.

(b) A gel is prepared by making the following formulations:

| | |
|---|---|
| Compound of formula (11) | 0.050 g |
| Erythromycin base | 4.000 g |
| Butylhydroxytoluene | 0.050 g |
| Hydroxypropylcellulose sold by the | 2.000 g |
| company Hercules under the name | |
| “Klucel HF” | |
| Ethanol (95°) qs | 100.000 g |

This gel is applied to a hypopigmented grafted skin one to three times a day for six to twelve weeks depending on the severity of the case treated.

(c) A lotion is prepared for correcting post-inflammatory hypopigmentation by mixing together the following ingredients:

| | |
|---|---|
| Compound of formula (2) | 0.030 g |
| Propylene glycol | 5.000 g |
| Butylhydroxy toluene | 0.100 g |
| Ethanol (95°) qs | 100.000 g |

This lotion is applied twice a day and a significant improvement is observed within a period of two to six weeks.

(d) A cosmetic composition to combat the harmful effects of sunlight is prepared by mixing together the following ingredients:

| | |
|---|---|
| Compound of formula (4) | 1.000 g |
| Benzylidenecamphor | 4.000 g |
| Fatty acid triglycerides | 31.000 g |
| Glyceryl monostearate | 6.000 g |
| Stearic acid | 2.000 g |
| Cetyl alcohol | 1.200 g |
| Lanolin | 4.000 g |
| Preserving agents | 0.300 g |
| Propylene glycol | 2.000 g |
| Triethanolamine | 0.500 g |
| Fragrance | 0.400 g |
| Demineralized water qs | 100.000 g |

This composition is applied daily and makes it possible to combat light-inducing aging.

(e) The following non-ionic oil-in-water cream is prepared:

| | |
|---|---|
| Compound of formula (3) | 0.500 g |
| Vitamin D3 | 0.020 g |
| Cetyl alcohol | 4.000 g |
| Glyceryl monostearate | 2.500 g |
| PEG-50 stearate | 2.500 g |
| Karite butter | 9.200 g |
| Propylene glycol | 2.000 g |
| Methyl para-hydroxybenzoate | 0.075 g |
| Propyl para-hydroxybenzoate | 0.075 g |
| Sterile deminealized water qs | 100.000 g |

This cream is applied to a skin affected with vitiligo once or twice a day for thirty days.

(f) A topical gel is prepared by mixing together the following ingredients:

| | | |
|---|---|---|
| Compound of formula (17) | 0.050 | g |
| Ethanol | 43.000 | g |
| α-Tocopherol | 0.050 | g |
| Carboxyvinyl polymer sold under | 0.500 | g |
| the name Carbopol 941 ® by | | |
| “Goodrich” | | |
| Triethanolamine as a 20% by | 3.800 | g |
| weight aqueous solution | | |
| Water | 9.300 | g |
| Propylene glycol qs | 100.00 | g |

This gel is applied to a skin affected with vitiligo one to three times a day for six to twelve weeks depending on the severity of the case treated.

(g) A hair lotion for repigmenting the hair is prepared by mixing together the following ingredients:

| | | |
|---|---|---|
| Compound of formula (9) | 0.05 | g |
| Compound sold under the | 1.00 | g |
| name Minoxidil ® | | |
| Propylene glycol | 20.00 | g |
| Ethanol | 34.92 | g |
| Polyethylene glycol | 40.00 | g |
| (molecular mass = 400) | | |
| Butylhydroxyanisole | 0.01 | g |
| Butylhydroxytoluene | 0.02 | g |
| Water | qs 100.000 | g |

This lotion is applied twice a day for three months to a scalp which has suffered considerable depigmentation.

(h) A post-cicatrization cream is prepared by mixing together the following ingredients:

| | | |
|---|---|---|
| Compound of formula (13) | 0.050 | g |
| Retinoic acid | 0.010 | g |
| Mixture of glyceryl stearate and | 15.000 | g |
| polyethylene glycol stearate | | |
| (70 mol) sold under the name | | |
| Gelot 64 ® by “Gattefosse” | | |
| Kernel oil polyoxyethylenated with | 8.000 | g |
| 6 mol of ethylene oxide, solder under | | |
| the name Labrafil M2130 CS ®, by | | |
| “Gattefosse” | | |
| Perhydrosqualene | 10.000 | g |
| Preserving agents | qs | |
| Polyethylene glycol (molecular | 8.000 | g |
| mass = 400) | | |
| Disodium salt of ethylenediamine- | 0.050 | g |
| tetraacetic acid | | |
| Purified water | qs 100.000 | g |

This cream is applied one to three times a day for six to twelve weeks.

(i) An oil-in-water cream is prepared by making the following formulation:

| | | |
|---|---|---|
| Compound of fommla (4) | 0.020 | g |
| Betamethasone 17-valerate | 0.050 | g |
| S-carboxymethylcysteine | 3.000 | g |
| Polyoxyethylene stearate (40 mol | 4.000 | g |
| of ethylene oxide) sold under the | | |
| name Myrj 52 ® by “Atlas” | | |
| Sorbitan monolaurate, polyoxy- | 1.800 | g |
| ethylenated with 20 ml of ethylene | | |
| oxide, sold under the name Tween | | |
| 20 ® by “Atlas” | | |
| Mixture of glyceryl mono- and | 4.200 | g |
| distearate sold under the name | | |
| Geleol ®, by “Gattefosse” | | |
| Propylene glycol | 10.000 | g |
| Butylhydroxyanisole | 0.010 | g |
| Butylhydroxytoluene | 0.020 | g |
| Cetostearyl alcohol | 6.200 | g |
| Preserving agents | qs | |
| Perhydrosqualene | 18.000 | g |
| Mixture of caprylic/capric | 4.000 | g |
| triglycerides sold under the | | |
| name Miglyol 812 ® by | | |
| “Dynamit Nobel” | | |
| Triethanolamine (99% by weight) | 2.500 | g |
| Water | qs 100.000 | g |

This cream is applied twice a day to a skin affected with pigmentation problems due to aging.

(j) The following cream of oil-in-water type is prepared:

| Lactic acid | 5.000 | g |
|---|---|---|
| Compound of formula (13) | 0.020 | g |
| Polyoxyethylene stearate (40 mol | 4.000 | g |
| of ethylene oxide) sold under the | | |
| name Myrj 52 ® by “Atlas” | | |
| Sorbitan monolaurate, polyoxy- | 1.800 | g |
| ethylenated with 20 mol of theylene | | |
| oxide sold under the name Tween | | |
| 20 ®, by “Atlas” | | |
| Mixture of glyceryl mono- and | | |
| distearate sold under the name | | |
| Celeol ®, by “Gattefosse” | 4.200 | g |
| Propylene glycol | 10.000 | g |
| Butylhydroxyanisole | 0.010 | g |
| Butylhydroxytoluene | 0.020 | g |
| Cetostearyl alcohol | 6.200 | g |
| Preserving agents | qs | |
| Perhydrosqualene | 18.000 | g |
| Mixture of caprylic/capric | 4.000 | g |
| triglycerides sold under the | | |
| name Miglyol 812 ® by | | |
| “Dynamit Nobel” | | |
| Water | qs 100.000 | g |
| (k) Dermal Lotion for spraying: | | |
| Compound of formula (15) | 5.000 | g |
| Ethanol | 30.000 | g |
| Demineralized water | qs 100.000 | g |
| (l) Hair lotion: | | |
| Compound of formula (18) | 3.000 | g |
| Propylene glycol | 30.000 | g |
| Ethyl alcohol | 40.500 | g |
| Water | qs 100.000 | g |

This lotion is applied to the scalp once or twice a day at a rate of 1 ml per application.

| (m) Thickened lotion: | | |
|---|---|---|
| Compound of formula (1) | 5.000 | g |
| Kawaine | 2.000 | g |
| Hydroxypropylcellulose | 3.500 | g |
| (Klucel G ® from Hercules) | | |
| Ethyl alcohol | qs 100.000 | g |

This thickened lotion is applied to the scalp once or twice a day at a rate of 1 ml per application.

| (n) Niosomal lotion: | | |
|---|---|---|
| Chimexane ML ® | 0.475 | g |
| Cholesterol | 0.475 | g |
| Monosodium stearoylglutamate | 0.050 | g |
| Compound of formula (3) | 0.100 | g |
| Preserving agents | qs | |
| Dyes | qs | |
| Fragrance | qs | |
| Demineralized water | qs 100.000 | g |

This lotion is applied to the scalp once or twice a day at a rate of 1 ml per application.

| (o) Lotion: | | |
|---|---|---|
| Compound of formula (17) | 5.000 | g |
| Propylene glycol monomethyl ether | 20.000 | g |
| (Dowanol PM ® from Dow Chemical) | | |
| Hydroxypropylcellulose (Klucel G ® | 3.000 | g |
| from Hercules) | | |
| Ethyl alcohol | 40.000 | g |
| Minoxidil | 2.000 | g |
| Water | qs 100.000 | g |

This thickened lotion is applied to the scalp once or twice a day at a rate of 1 ml per application.

While the invention has been described with respect to certain specific embodiments, it will be appreciated that many modifications and changes thereof may be made by those skilled in the art without departing from the spirit of the invention. It is intended, therefore, by the appended claims to cover all modifications and changes that fall within the true spirit and scope of the invention.

What is claimed is:

1. A cosmetic, dermatological, and/or therapeutic method for altering (promoting or inhibiting) the pigmentation of human skin and/or hair of a human subject comprising topically applying an effective amount of at least one substituted aromatic or heterocyclic carboxylic acid, derivative or pharmaceutically acceptable salt thereof for a sufficient length of time to induce skin and/or hair pigmentation, with the proviso that said carboxylic acid does not comprise a phenol, naphthol, thiophenol or thionaphthol function, either free or protected by a protective group.

2. The method of claim 1, wherein said compound is a retinoid signaling molecule that promotes pigmentation of human skin and/or hair.

3. The method of claim 1, wherein said compound is selected from the group consisting of:
4-[1-(5,6.7,8-tetrahydro-5,5,8,8-tetramethyl-2-naphthyl)ethenyl]benzoic acid;
3-[3-(5,5,8,8-tetramethyl-5,6,7,8-tetrahydro-naphthalen-2-yl)-phenyl]-acrylic acid;
3-[3-(3,5.5,8,8-pentamethyl-5,6,7,8-tetrahydro-naphthalen-2-yl)-phenyl]-but-2-enoic acid;
6-[1-(3,5,5,8,8-pentamethyl-5,6,7,8-tetrahydro-naphthalen-2-yl)-cyclopropyl]-nicotinic acid;
3-[3-(3,5,5,8,8-pentamethyl-5.6,7.8-tetrahydro-naphthalen-2-yl)-phenyl]-acrylic acid;
4-(3,5,5,8,8-pentamethyl-5,6,7,8-tetrahydro-naphthalene-2-carbonyl)-benzoic acid;
4-(3-bromo-5,5,8,8-tetramethyl-5,6,7,8-tetrahydro-naphthalen-2-yloxy)-benzoic acid;
5-[(E)-2-(3,5,5,8,8-pentamethyl-5,6,7,8-tetrahydro-naphthalen-2-yl)-propenyl]-thiophene-3-carboxylic acid;
3-[3-(5,5,8,8-tetramethyl-5,6,7,8-tetrahydro-naphthalen-2-ylsulfanyl)-phenyl]-acrylic acid;
6-(3-butyl-5,5,8,8-tetramethyl-5,6,7,8-tetrahydro-naphthalen-2-ylsulfanyl)-nicotinic acid;
4-(3,5,5,8,8-pentamethyl-5,6,7,8-tetrahydro-2-naphthylthio) benzoic acid;
3-(3,5,5,8,8-pentamethyl-5,6,7,8-tetrahydro-2 naphthyl)phenyl propiolic acid; and
6-(3,5,5,8,8-pentamethyl-5,6,7,8-tetrahydro-2 naphthylthio) nicotinic acid.

4. The method of claim 1, which is used to promote the browning or tanning of human skin.

5. The method of claim 1, which is used to promote coloration of human hair.

6. The method of claim 1, wherein said compound is contained in a topically applicable composition that comprises from 0.001 to 30% by weight of said compound relative to the weight of the composition.

7. The method of claim 1, wherein the amount of said compound ranges from 0.5 to 15% by weight of the composition.

8. A pharmaceutical/cosmetic/dermatological composition that alters the pigmentation of human skin and/or hair upon topical application that comprises
(i) an amount of at least one substituted aromatic or heterocyclic carboxylic compound, derivative, or pharmaceutically acceptable salt thereof, with the proviso that said compound does not contain a free or protected phenol, naphthol, thiophenol or thionaphthol group which is effective to alter the pigmentation or human skin and/or hair; and
(ii) a pharmaceutically, cosmetically, or dermatologically acceptable carrier.

9. The composition of claim 8, wherein said compound is selected from the group consisting of:
4-[1-(5,6.7,8-tetrahydro-5,5,8,8-tetramethyl-2-naphthyl)ethenyl]benzoic acid;
3-[3-(5,5,8,8-tetramethyl-5,6,7,8-tetrahydro-naphthalen-2-yl)-phenyl]-acrylicacid;
3-[3-(3,5.5,8,8-pentamethyl-5,6,7,8-tetrahydro-naphthalen-2-yl)-phenyl]-but-2-enoic acid;
6-[1-(3,5,5,8,8-pentamethyl-5,6,7,8-tetrahydro-naphthalen-2-yl)-cyclopropyl]-nicotinic acid;
3-[3-(3,5,5,8,8-pentamethyl-5.6,7.8-tetrahydro-naphthalen-2-yl)-phenyl]-acrylic acid;
4-(3,5,5,8,8-pentamethyl-5,6,7,8-tetrahydro naphthalene-2-carbonyl)-benzoic acid;
4-(3-bromo-5,5,8,8-tetramethyl-5,6,7,8-tetrahydro-naphthalen-2-yloxy)-benzoic acid;
5-[(E)-2-(3,5,5,8,8-pentamethyl-5,6,7,8-tetrahydro-naphthalen-2-yl)-propenyl]-thiophene-3-carboxylic acid;
3-[3-(5,5,8,8-tetramethyl-5,6,7,8-tetrahydro-naphthalen-2-ylsulfanyl)-phenyl]-acrylic acid;
6-(3-butyl-5,5,8,8-tetramethyl-5,6,7,8-tetrahydro-naphthalen-2-ylsulfanyl)-nicotinic acid;
4-(3,5,5,8,8-pentamethyl-5,6,7,8-tetrahydro-2-naphthylthio) benzoic acid;
3-(3,5,5,8,8-pentamethyl-5,6,7,8-tetrahydro-2 naphthyl)phenyl propiolic acid; and
6-(3,5,5,8,8-pentamethyl-5,6,7,8-tetrahydro-2 naphthylthio) nicotinic acid.

10. The composition of claim 8, wherein the amount of said compound ranges from 0.001 to 30% by weight relative to the weight of the compositions.

11. The composition of claim 8, wherein the amount of said compound ranges from 0.5 to 15% by weight relative to the weight of the composition.

12. The composition of claim 8, which is in a form suitable for topical application selected from the group consisting of a cream, a milk, a cream-gel, a fluid lotion, an oil-in-water emulsion, a water-in-oil emulsion, and a vesicle dispersion.

13. The composition of claim 8, which comprises another compound which affects skin and/or hair pigmentation.

14. The composition of claim 8, which further comprises at least one substituent selected from the group consisting of ionic or non-ionic thickeners, softeners, antioxidants, opacifiers, stabilizers, emollients, organic sunscreens which are active in the UV-A and/or UV-B region, photoprotective inorganic and nanopigments, moisturizers, vitamins, fragrances, preservatives, fillers, sequestering agents, dyestuffs, and colorants.

15. An in vitro method for selecting a compound or combination of compounds that alters (increases or decreases) pigmentation of human skin and/or hair comprising the following steps:
(i) incubating at least two melanocyte cultures with a compound or combination of compounds, the effect of which on pigmentation of human skin and/or hair is to be evaluated;
(ii) measuring the effect of said compound, or combination of compounds, on the proliferation of melanocytes contained in one of said cultures relative to a control melanocyte culture which has been cultured under the same conditions as said culture except in the absence of said compound or combination of compounds;
(iii) concurrently or substantially concurrently to step (ii), measuring the melanin content of cells contained in a second of said cultures which has been incubated with said compound or combination of compounds and comparing said melanin content to a control culture grown under the same conditions but in the absence of said compound or combination of compounds;
(iv) comparing the data obtained in steps (ii) and (iii) and normalizing said data based on the proliferation (number of cells) in said melanocyte cultures in order to determine the effect, if any, of said compound, or combination of compounds, on melanin production by melanocytes.

16. The method of claim 15, wherein said melanocyte cultures comprise human melanocytes.

17. The method of claim 16, wherein said human melanocytes are obtained from neonatal skin.

18. The method of claim 15, wherein step (ii) comprises determining the proliferation of melanocytes in said first culture by absorbence.

19. The method of claim 18, wherein step (ii) comprises treating said cells with an (MTS) 3-(4,5-dimethylthiazol-2-yl)-5-(3-carboxymethoxyphenyl)-2-(4-sulfophenyl)-2H-tetrazolium solution and determining the absorbence of said MTS-treated cells at 490 nm.

20. The method of claim 15, where in step (iii) melanin content is determined by lysing the cells, treating the cells with an alkaline solution, and measuring the absorbence at 405 nm to determine the amount of alkali-soluble melanin contained therein.

21. The method of claim 15, wherein said compound or combination of compounds is selected on the basis that it alters melanin content by at least 20% relative to a control melanocyte culture which is not treated with said compound or combination of compounds.

22. The method of claim 21, wherein said compound or combination of compounds alters melanin content relative to a control melanocyte culture by at least 50%.

23. The method of claim 22, wherein said compound or combination of compounds alters melanin content relative to a control melanocyte culture by at least 150%.

24. The method of claim 22, wherein said compound or combination of compounds alters melanin content by at least 250%.

25. The method of claim 1, wherein said compound displays activity for RXRs.

26. The composition of claim 8, wherein said compound displays activity for RXRs.

27. The method of claim 25, wherein said compound exhibits a bind for RXR of less than 5000 nM.

28. The composition of claim 26, wherein said compound exhibits a binding for RXR of less than 5000 nM.
